(19) United States
(12) Patent Application Publication
(10) Pub. No.: US 2022/0370297 A1
(43) Pub. Date: Nov. 24, 2022

(54) GASTRIC PLACEMENT CONFIRMATION DEVICE

(71) Applicant: Regents of the University of Minnesota, Minneapolis, MN (US)

(72) Inventors: Jonathan Strutt, Edina, MN (US); Vidhu Shekar Pandey, Minneapolis, MN (US); Jeffrey P. Louie, Minneapolis, MN (US)

(21) Appl. No.: 17/664,339

(22) Filed: May 20, 2022

Related U.S. Application Data
(60) Provisional application No. 63/201,960, filed on May 20, 2021.
(60) Provisional application No. 63/201,959, filed on May 20, 2021.

(51) Int. Cl. A61J 15/00

(52) U.S. Cl. CPC A61J 15/0084; A61J 15/0015; A61J 15/0003; A61M 25/0102

(57) ABSTRACT

Devices, systems, and methods for inserting a medical device, such as a gastrostomy tube, are described. For example, a device includes a housing defining an outer surface, a first end, and a second end, the housing comprising an inner passage that extends from the first end of the housing to the second end of the housing, wherein the outer surface defines an aperture in fluid communication with a portion of the inner passage, and a sensing device configured to cover the aperture, the sensing device configured to detect a property of fluid within the inner passage.

This application claims the benefit of U.S. Provisional Patent Application Nos. 63/201,960, filed May 20, 2021, and U.S. Provisional Patent Application No. 63/201,959, filed May 20, 2021, the entire content of each application is incorporated herein by reference.

### TECHNICAL FIELD

The invention relates to medical devices, and more specifically, devices that aid in the placement of medical devices.

### BACKGROUND

Due to trauma, disease, or other causes, medical tubing or other devices that permit the flow of fluid into or out of a patient may need to be inserted into a patient. For example, a medical device called a gastrostomy tube may be placed through the skin and into the stomach to provide a fluid passageway into the stomach. The gastrostomy tube may be called a feeding port because it can enable direct delivery of fluids and/or food directly into the patient's stomach. In other examples, a nasogastric tube may be placed through the nasal or oral passage and into the stomach of the patient in order to deliver nutrition and/or medication to the patient.

### SUMMARY

This disclosure describes devices, systems, and techniques for confirming the placement of a medical device, such as a nasogastric tube, orogastric tube, or gastrostomy tube, at least partially within a target anatomical location of a patient. For example, a device may include a housing that defines an inner passage configured to direct fluid from a first end of the housing to a second end of the housing. The first end of the housing may be configured to be coupled to (e.g., attached to and/or inserted within) a portion of the medical device, such as a nasogastric tube, to enable the device to draw fluid from the medical device and into the inner passage of the housing. The housing of the device includes an aperture that provides fluid communication from a portion of the inner passage through an outer surface of the housing.

A sensing device (e.g., pH paper or other material or device), can be configured to cover the aperture. The second end of the housing may be configured to couple with a suction device, such as a syringe, that can be operated to generate a vacuum within the inner passage which causes fluid within the anatomical location (e.g., the lumen of the stomach) of the patient to be drawn through the inner passage, through the aperture, and contact the sensing device. The sensing device may indicate a property of the fluid, such as a pH of the fluid, which the user can utilize to determine proper placement of the medical device within the target anatomical location of the patient.

In one example, a device includes a housing defining an outer surface, a first end, and a second end, the housing comprising an inner passage that extends from the first end of the housing to the second end of the housing, wherein the outer surface defines an aperture in fluid communication with a portion of the inner passage; a stylet coupled to the first end of the housing, the stylet defining a channel in fluid communication with the inner passage; and a sensing device configured to cover the aperture, the sensing device configured to detect a property of fluid within the inner passage.

In another example, a method includes inserting a stylet of a device into an inner passage of a gastrostomy tube, wherein the device comprises a housing defining an outer surface, a first end, and a second end, the housing comprising an inner passage that extends from the first end of the housing to the second end of the housing, wherein the outer surface defines an aperture in fluid communication with a portion of the inner passage; the stylet coupled to the first end of the housing, the stylet defining a channel in fluid communication with the inner passage; and a sensing device configured to cover the aperture, the sensing device configured to detect a property of fluid within the inner passage; inserting the stylet and a distal portion of the gastrostomy tube into a stomach of a patient; withdrawing fluid from the stomach, through the stylet, and through the inner passage of the device past the aperture; and confirming, via the sensing device, that the property of the fluid is consistent with contents of the stomach.

In another example, a system includes a device that includes a housing defining an outer surface, a first end, and a second end, the housing comprising an inner passage that extends from the first end of the housing to the second end of the housing, wherein the outer surface defines an aperture in fluid communication with a portion of the inner passage; a stylet coupled to the first end of the housing, the stylet defining a channel in fluid communication with the inner passage; and a sensing device configured to cover the aperture, the sensing device configured to detect a property of fluid within the inner passage; and a syringe configured to couple to the second end of the housing, wherein the syringe is configured to withdraw fluid through the inner passage and to the aperture in response to withdrawal of a plunger within the syringe.

In another example, a device includes a housing defining an outer surface, a first end, and a second end, the housing comprising an inner passage that extends from the first end of the housing to the second end of the housing, wherein the outer surface defines an aperture in fluid communication with a portion of the inner, and wherein the inner passage is curved outward from a central axis of the housing to approach the outer surface and the aperture; a coupling mechanism configured to couple the first end of the housing to a medical device at least partially implantable within a patient; and a sensing device configured to cover the aperture, the sensing device configured to detect a property of fluid within the inner passage.

In another example, a method includes coupling a first end of a device to a nasogastric tube, wherein the nasogastric tube is at least partially implantable within a patient, wherein the device comprises: a housing defining an outer surface, a first end, and a second end, the housing comprising an inner passage that extends from the first end of the housing to the second end of the housing, wherein the outer surface defines an aperture in fluid communication with a portion of the inner passage, and wherein the inner passage is curved outward from a central axis of the housing to approach the outer surface and the aperture; a coupling mechanism configured to couple the first end of the housing to the nasogastric tube; and a sensing device configured to cover the aperture, the sensing device configured to detect a property of fluid within the inner passage; withdrawing fluid from the stomach, through the nasogastric tube, and through the inner passage of the device past the aperture; and confirming, via the sensing device, that the property of the fluid is consistent with contents of the stomach.

In another example, a system includes a device that includes a housing defining an outer surface, a first end, and a second end, the housing comprising an inner passage that extends from the first end of the housing to the second end of the housing, wherein the outer surface defines an aperture in fluid communication with a portion of the inner passage, and wherein the inner passage is curved outward from a central axis of the housing to approach the outer surface and the aperture; a coupling mechanism configured to couple the first end of the housing to a medical device at least partially implantable within a patient, the sensing device configured to detect a property of fluid within the inner passage; and a syringe configured to couple to the second end of the housing, wherein the syringe is configured to withdraw fluid through the inner passage and to the aperture in response to withdrawal of a plunger within the syringe.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1A is a conceptual diagram of an example system configured to confirm a position a medical device and check for proper placement within the patient, in accordance with one or more aspects of this disclosure.

FIG. 1B is a conceptual diagram of an example system configured to confirm a position a medical device and check for proper placement within the patient, in accordance with one or more aspects of this disclosure.

FIG. 2 is a cross-sectional view of an example device configured to confirm a position a medical device and check for proper placement within the patient.

FIG. 3 is an image of an example device in conjunction with a syringe and a gastrostomy button for placement within a patient.

FIG. 4 is an image of the example device of FIG. 3.

FIG. 5 is an image of the example device of FIG. 3 with the shaft of the device inserted into the tube of the gastrostomy button.

FIG. 6 is an image of an example device attached to a syringe and a gastrostomy button for placement within a patient.

FIG. 7 is a cross-sectional view of an example device having a curved inner passage.

FIG. 8 is a cross-sectional view of an example device having two curved portions of an inner passage in fluid communication with respective apertures at the outer surface of the housing the device.

FIG. 9 is a flowchart of an example technique for using a device for positioning a medical device in a patient, in accordance with one or more techniques of this disclosure.

FIG. 10 is a flowchart of an example technique for using a device for confirming the position of a medical device in a patient, in accordance with one or more techniques of this disclosure.

### DETAILED DESCRIPTION

As described herein, methods, systems, and devices may enable confirmation of positioning of a medical device, such as a gastrostomy tube, nasogastric tube, or orogastric tube, into a desired location of the patient. It may be difficult for users (e.g., clinicians, patients, or caregivers) to properly position medical devices at target locations within the patient. For example, gastric medical devices, such as a gastrostomy tube or nasogastric tube, may need to be positioned such that a distal portion of the gastrostomy tube or nasogastric tube extends into the stomach to facilitate stomach contents removal and/or food or fluid delivery into the stomach. Herein, the term gastrostomy tube may refer to any medical device that is configured to be positioned to provide a passage from the stomach and outward to the exterior of the patient. A gastrostomy tube may include a gastrostomy button or low profile gastrostomy tube in some examples.

In one example, when inserting a gastrostomy tube through a stoma, there is a direct relationship between how long the previous distal end of the gastrostomy button has been out and the difficulty of the reinsertion of the same or other low-profile gastrostomy tube (e.g., a gastrostomy button). The longer the time between the tube being accidentally dislodged by the patient and the attempt at reinsertion, the more difficult the reinsertion typically is. There are many methods of reinsertion including manually placing a new low-profile gastrostomy tube of the same diameter, placement of a smaller diameter size until a larger once can be inserted at a later time, progressive dilation of the stoma tract with a set of Hager dilators in order to accommodate the correct size, or insertion of a shaft to allow for a more rigid tube which can accept the increased forced required to insert the low-profile gastrostomy tube through the tight stoma. When inserting a nasogastric tube or orogastric tube, the user needs to ensure that the distal end of the tube is inserted within the stomach in order to deliver food or medication and/or remove stomach contents, for example.

Confirmation of the low-profile gastrostomy tube or nasogastric tube placement is then performed by aspirating fluid through the low-profile gastrostomy tube or nasogastric tube. Stomach content confirmation is assumed by the color of fluid and the ease of flow through the inner passage of the low-profile gastrostomy tube or nasogastric tube. However, if a false tract was created by the reinsertion of the tube, the user may be falsely reassured into thinking that the placement of the tube was adequate. The aspiration of fluid such as serosanguinos, cystic, or infectious pus may mislead the provider into thinking that the placement is correct when it is, in fact, not. For gastrostomy tubes, malposition (or inadvertent removal) of percutaneous gastrostomy (PEG) tubes are the chief PEG tube related complaint in the Pediatric emergency department. For nasogastric tubes, improper insertions can lead to inadequate medication or nutrition delivery, inadequate removal of stomach contents, or inadvertent administration of fluids, nutrition, or medication to undesired locations such as the lungs, causing great harm to the patient. In this manner, positioning of a gastrostomy tube, nasogastric tube, or other medical device can be difficult to complete correctly.

This disclosure describes devices, systems, and techniques for confirming the correct position a medical device, such as a low-profile gastrostomy tube or nasogastric device, at least partially within a target anatomical location (e.g., a target lumen of a stomach or other void or position within an anatomical structure) of a patient. For example, a device may include a housing that defines an inner passage configured to direct fluid from a first end of the housing to a second end of the housing. In one example, a coupling mechanism, such as a shaft or luer connector, may be attached to the first end of the housing and configured to be coupled to a portion of the medical device, such as the low-profile gastrostomy tube or nasogastric tube, to enable suction or vacuum from the device to the medical device. For an example of low-profile gastrostomy tubes, the shaft may be configured to aid in replacement of Mic-Key® brand low-profile gastrostomy tubes or any other brand, size, or design of low-profile gastrostomy tubes. The shaft can aid in difficult low-profile gastrostomy tube insertions in the emergency department, clinic, or home by providing increased rigidity for the stomas which have begun to close and are tight. A luer connector or other threaded or friction fit connector can be used to provide a fluid-sealed connection between the device and medical device, such as a nasogastric tube.

The housing of the device may also include an aperture that provides fluid communication from a portion of the inner passage through an outer surface of the housing. The inner passage may be curved outward from a central axis of the housing to approach the outer surface and the aperture. A sensing device (e.g., pH paper or other material or device), can be configured to cover the aperture. The second end of the housing may be configured to couple with a suction device, such as a syringe, that can be operated to generate a vacuum within the inner passage which causes fluid within the lumen of the patient to be drawn through the inner passage, through the aperture, and contact the sensing device. The sensing device may indicate a property of the fluid, such as a pH of the fluid, which the user can utilize to determine proper placement of the medical device within the target lumen of the patient.

In some examples, the sensing device may include a pH strip placed onto the outer surface of the housing of the device such that the pH strip can aid in confirming correct placement of the low-profile gastrostomy tube or nasogastric tube into the stomach. When the user has inserted the low-profile gastrostomy tube or nasogastric tube correctly and withdraws plunger of the syringe, stomach contents will be pulled into the device and the pH strip will register the acidity of the stomach contents, confirming correct, or incorrect, placement. In this manner, the user can be certain of correct low-profile gastrostomy tube or nasogastric tube placement instead of guess based on color of the contents removed from the stomach. In addition, as described below, the device may be configured to sense multiple different properties of the fluid to further identify the actual location of the distal end of the low-profile gastrostomy tube or nasogastric tube.

Although the medical device is generally described herein as a gastrostomy tube or nasogastric tube, other types of medical devices may be positioned with the device in other examples. For example, the shaft and/or sensing device of the device may be used to aid in the positioning of other gastric tubes, urinary tubes, fecal tubes, peritoneal devices, vascular devices, etc. In some examples, the medical devices described here may be configured to be connected to one or more catheters configured to be inserted into the patient for sampling urine, blood, fluid from bronchi (e.g., via a bronchoscope), or any other bodily fluid. In this manner, when fluid is withdrawn from the patient via the catheter, the medical device can use a chemical reactive paper or other material to confirm one or more characteristics of the fluid withdrawn through the catheter. In one example, the medical device may be configured to detect jejunal fluid from a catheter or other tube to confirm that the catheter or tube is disposed within the small intestine.

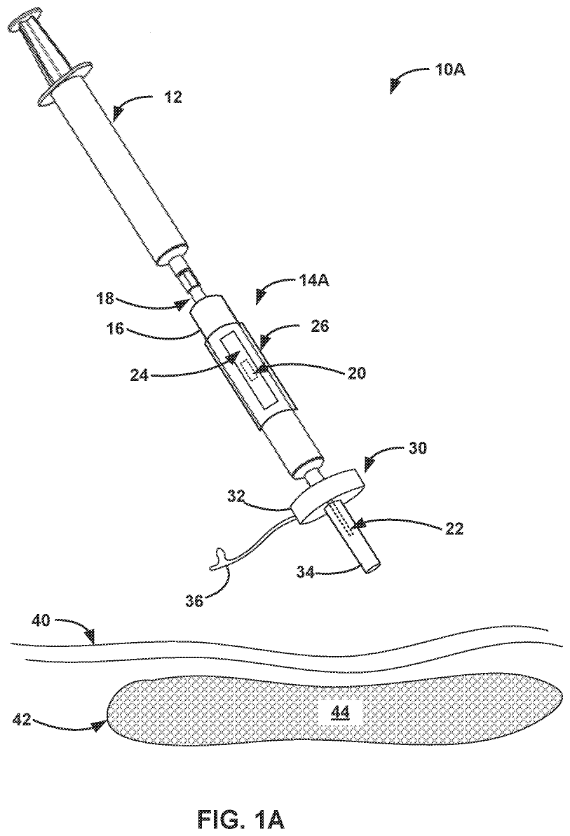

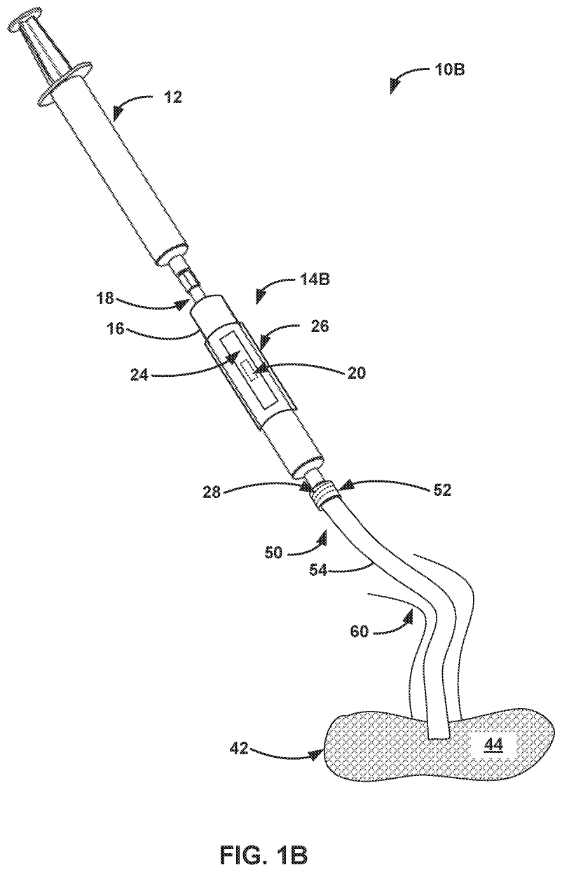

FIG. 1A is a conceptual diagram of an example system 10B configured to position a medical device 30 and check for proper placement within the patient, in accordance with one or more aspects of this disclosure. As shown in example FIG. 1A, medical device 30 is in the form of a low-profile gastrostomy tube (e.g., a gastrostomy button) designed to be used as a port to the stomach 42 of a patient. Device 14A includes a housing 16, a luer connector 18 at a proximal end of housing 16, a shaft 22 at a distal end of housing 16, sensing device 24, and covering 26 that retains sensing device 24 against the outer surface of housing 16. Housing 16 also defines aperture 20 that provides fluid communication between an inner passage within housing 16 and the sensing device 24 covering aperture 20. Shaft 22 can be inserted within tube 34 of medical device 30, and medical device 30 also includes proximal hub 32 and plug 36. Syringe 12 is shown connected to luer connector 18 and may draw fluid 44 into device 14A by withdrawing the plunger of syringe 12. However, syringe 12 may be coupled to device 14A after device 14A has been used to insert tube 34 of medical device 30 through skin 40 and into the target location, such as stomach 42.

As described above, the inadvertent removal of a medical device 30, such as a skin-level gastrostomy button (G-button), may be the most common complaint associated with gastrostomy tube evaluations in the Pediatric Emergency Department (ED). There are many reasons that make replacement of the G-button less than straightforward, including both duration of G-button dislodgement and patient cooperation. System 10B and/or device 14A may be configured to improve the ease of insertion for patients presenting with G-button dislodgement or similar issues. In addition, device 14A may aid families in attempting to replace G-buttons at home and avoid presentation to the ED.

In some examples, the replacement of a G-button into a mature stoma tract may be more difficult than expected depending on factors such as duration of dislodgement and patient cooperation. ED providers may use a rigid shaft such as an inverted cotton applicator to stiffen the flexible tubing of the G-button and aid in replacement. Such a method requires the operator to be aware of the exact position of the applicator so that inadvertent tissue damage does not occur, and there is no good way of verifying placement until it is fully in position with the balloon dilated. Device 14A and system 10B and related examples described herein can provide advantages such as improved first attempt G-button replacement for providers and more likely replacement at home to avoid unnecessary trips to the ED.

In one example, system 10B and associated techniques may aid in the reinsertion of accidentally dislodged low-profile gastrostomy tube devices, such as the Mic-Key® low-profile gastrostomy tube. Shaft 22 may facilitate reinsertion of the low-profile gastrostomy tube through difficult to insert or “tight” stomas caused by prolonged time without the distal end of the low-profile gastrostomy tube in place and/or provide a friction fit that facilitates fluid communication between device 14A and medical device 30. In some examples, shaft 22 can be more rigid than tube 34 of medical device 30 such that shaft 22 can provide additional, or increased, rigidity to allow for an increased force to be applied when inserting tube 34 without bending tube 34 of medical device 30, thus aiding in insertion through a tight stoma. In other example, shaft 22 may be a flexible tube configured to mate with tube 34 and provide fluid communication between device 14A and medical device 30. Device 14A may also incorporate sensing device 24, such as an embedded pH strip, in or on housing 16 such that when stomach 42 contents are aspirated through device 14A to confirm placement, the pH test strip will change color, indicating the acidity of the fluid 44 withdrawn therein. The color on the strip will indicate whether or not the fluid is consistent with the acidity expected of stomach 42 contents. Other example sensing materials may include gastric paper, litmus paper, a pepsin assay, or any other types of sensing materials. In other examples, sensing device 24 may be constructed of materials that detect one or more specific chemicals, temperatures, fluids, etc., associated with the target location for medical device 30. As described herein, system 10B and device 14A may be used by medical professionals (e.g., a physician, nurse, or other clinician) or even non-professional caregivers (e.g., relatives or friends) of the patient.

Device 14A may include housing 16 defining an outer surface, a first end, and a second end, where housing 16 includes an inner passage (not shown) that extends from the first end of the housing to the second end of the housing. The outer surface of housing 16 may define aperture 20 that is in fluid communication with a portion of the inner passage. Shaft 22 may be coupled to the first end of housing 18, where shaft 22 defines a channel in fluid communication with the inner passage. In addition, device 14A includes sensing device 24 configured to cover aperture 20. Sensing device 24 may be configured to detect a property of fluid 44 within the inner passage of housing 16.

In some examples, device 14A includes cover 26 that retains sensing device 24 against outer surface of housing 16 and aperture 20. For example, cover 26 may be a flexible polymer (e.g., plastic wrap or heat-shrink wrap) that can be wrapped around housing 16. Cover 26 may be transparent or semi-transparent to enable the user to visualize any changes to sensing device 24. In some examples, cover 26 may be a rigid cover that snaps or includes one or more hinges in order to close cover 26 over sensing device 24.

Sensing device 24 may include a material that changes when in contact with fluid 44 once passing through aperture 20. For example, sensing device 24 may be a strip of pH paper configured to change color upon exposure to fluids of certain pH. In other examples, sensing device 24 may be or include immunochromatographic paper configured to detect a specific enzyme (e.g., pepsin located in the gastrointestinal tract to break down proteins) or other chemical. In this manner, any paper or other sensor configured to chemically react to fluids at aperture 20 may be used as or a part of sensing device 24. In other examples, sensing device 24 may include an electronic device or other component configured to provide an indication of one or more components contained within the fluid that comes in contact with sensing device 24 through aperture 20.

Fluid drawn into device 14A flows through one or more inner passages within housing 16. In some examples, the inner passage is defined by one or more inner walls of housing 16. In other examples, housing 16 includes one or more tubes disposed within an inner wall of the housing. The one or more tubes then define part or all of the inner passage. The inner passage may extend from the distal end of housing 16 through the proximal end of housing 16. Aperture 20 is in fluid communication with a portion of the inner passage. In examples with multiple inner passages, some or all of the inner passages may be fluid communication with respective different apertures on the external surface of housing 16.

In some examples, housing 16 generally defines a cylindrical shape, where the outer surface of housing 16 is the curved portion of the cylindrical shape. Housing 16 may be formed to different shapes, such as rectangular, ovoid, or irregular shapes. Housing 16 may be constructed of one or more materials, such as a polymer, polymer resin (e.g., BioMed Clear), composite, metal or metal alloy, or any other material. Housing 16 may be injection molded, extruded, formed, cast, milled, formed from two or more pieces and attached, or any other manufacturing method. In one example, housing 16 may be 3D printed using a thermoplastic polymer such as polylactic acid (PLA) or any other material that can be printed. Generally, housing 16 and/or the walls of the inner passage may be constructed of a biocompatible material, but non biocompatible materials may be used in other examples (e.g., if device 14A is configured to be disposable or a single-use device). In some examples, device 14A (including housing 16 and shaft 22, for example) may be constructed of one or more materials configured to be sterilizable for multiple uses.

Shaft 22 may have a diameter, length, and shape configured to be inserted into certain medical devices for placement in the patient. For example, shaft 22 may be configured to be inserted into tube 34 of medical device 30, which may be a low-profile gastrostomy tube. Shaft 22 may be configured to be more rigid than tube 34 of medical device 30 in order to promote insertion of tube 34 through skin 40 and into stomach 42 of the patient. In one example, the outer diameter of shaft 22 may be approximately 2.5 mm, and the inner diameter of shaft 22 may be approximately 1.5 mm. The diameter of the inner passage may be approximately 2 mm in this example. However, diameters may be smaller or larger than these examples. Shaft 22 may be constructed of a material such as stainless steel, brass, carbon fiber, a polymer, or any other metal alloy or composite material. Shaft 22 may be epoxied or otherwise adhered or screwed into housing 16. Similarly, luer connector 18 may be epoxied or otherwise adhered or screwed into housing 16.

Device 14A may be configured with different sizes of shaft 22. The size of shaft 22 may be selected to correspond to different medical devices 30, such as different lengths and/or inner diameters of tube 34. In one example, tube 34 may be between approximately 1.0 cm and 2.5 cm for a low-profile gastrostomy tube. A low-profile gastrostomy tube may also have a balloon at the distal end of tube 34 that, when inflated, increases the diameter of the low-profile gastrostomy tube to contact the inner wall of stomach 42 and prevent the low-profile gastrostomy tube from being removed from stomach 42. In some examples, housing 16 may include a pressurization channel that can enable a second syringe to be attached to housing 16 and add air or fluid through the pressurization channel and into the balloon at the distal end of tube 34. Plug 36 may be used to close the proximal opening of tube 34 when positioned within the patient to prevent fluid 44 from exiting tube 34.

In operation of inserting a low-profile gastrostomy tube as medical device 30, a user may slip medical device 30 into shaft 22. In some examples, a lubricant may be used to facilitate insertion of shaft 22 into tube 34 of medical device 34. The user may also attach syringe 12 to luer connection 18 of device 14A. The user may then insert tube 34 through skin 40, past the surface of stomach 42 through an existing stoma, and into place partially within stomach 42. At this time, the user can withdraw the plunger from syringe 12 to create a vacuum in syringe 12 to cause fluid 44 to be drawn through device 14A and aspirated from stomach 42. As fluid 44 is drawn through the inner passage of housing 16, some fluid will pass through aperture 20 and make contact with sensing device 24 that covers at least a portion of aperture 20. The user can then confirm that the fluid 44 within the inner passage is indeed contents of stomach 42 to ensure correct placement of tube 34. For example, if sensing device 24 is pH paper, the pH paper will turn color (e.g., turn yellow or orange) indicting the fluid is acidic. Once the user confirms correct placement of tube 34 of low-profile gastrostomy tube, the user can inflate the balloon of the low-profile gastrostomy tube to hold it in place and remove shaft 22 from the low-profile gastrostomy tube while the low-profile gastrostomy tube remains in place.

Although device 14A is generally described for use as a low-profile gastrostomy tube placement device, device 14A may be used to position other medical devices and/or confirm correct placement by aspirating fluid through device 14A.

FIG. 1B is a conceptual diagram of an example system 10B configured to confirm a position medical device 50 and check for proper placement within the patient, in accordance with one or more aspects of this disclosure. System 10B is substantially similar to system 10A of FIG. 1A. For example, device 14B is substantially similar to device 14A of FIG. 1A, but device 14B is configured to couple with medical device 50 using a different mechanism than shaft 22 described with respect to device 14A.

As shown in the example of FIG. 1B, device 14B includes housing 16, luer connector 18 at a proximal end of housing 16, threaded connector 28 at a distal end of housing 16, sensing device 24, and covering 26 that retains sensing device 24 against the outer surface of housing 16. Housing 16 also defines aperture 20 that provides fluid communication between an inner passage within housing 16 and the sensing device 24 covering aperture 20. Threaded connector 28 may be configured to mate with threaded connector 52 of medical device 50. Threaded connector 28 may be part of a luer connector or other type of connector utilizing a helical structure to secure device 14B with medical device 50. In other examples, device 14B may utilize a different coupling mechanism than threaded connector 28, such as a friction fit, one or more latches, or any other mechanical structure configured to secure the distal end of housing 16 with medical device 50. Syringe 12 is shown connected to luer connector 18 and may draw fluid 44 into device 14A by withdrawing the plunger of syringe 12.

In response to connecting medical device 50 and device 14B via threaded connector 28 and threaded connector 52, the inner passage of device 14B may be in fluid communication with the lumen that runs the length of tube 54 of medical device 50. For example, medical device 50 may be a nasogastric tube that includes threaded connector 52 (e.g., a male or female portion of a luer connector) coupled to tube 54. Tube 54 may be configured with a length sufficient to extend from inside stomach 42, through esophagus 60 and out of the nose of the patient.

Medical device 50 may be a gastric tube, such as a nasogastric tube. A clinician may initially insert tube 54 through the nasal passage of the patient, through esophagus 60, and into stomach 42 such that the distal end of tube 54 is disposed within fluid 44 of stomach 42. When performing this procedure, the clinician may need to withdraw fluid 44 out from stomach 42 in order to confirm that tube 54 is correctly placed (or re-placed) within stomach 42. However, it may not be evident from removed contents that tube 54 is actually within stomach 42. For example, the fluid removed can include blood, saliva, or other contents that may not be identifiable as stomach contents or not. Similar to device 14A, device 14B may also incorporate sensing device 24, such as an imbedded pH strip, in or on housing 16 such that when stomach 42 contents are aspirated through device 14B to confirm placement, the pH test strip will change color, indicating the acidity of the fluid 44 withdrawn therein. The color on the strip will indicate whether or not the fluid is consistent with the acidity expected of stomach 42 contents. In other examples, sensing device 24 may be constructed of materials that detect one or more specific chemicals, temperatures, fluids, etc., associated with the target location for medical device 30. As described herein, system 10B and device 14B may be used by medical professionals (e.g., a physician, nurse, or other clinician) or even non-professional caregivers (e.g., relatives or friends) of the patient.

Although device 14B is generally described for use as a nasogastric tube, device 14B may be used to position other medical devices and/or confirm correct placement by aspirating fluid through device 14B. These devices can be used in conjunction with the stomach, other gastrointestinal organ, or any other target location within a patient.

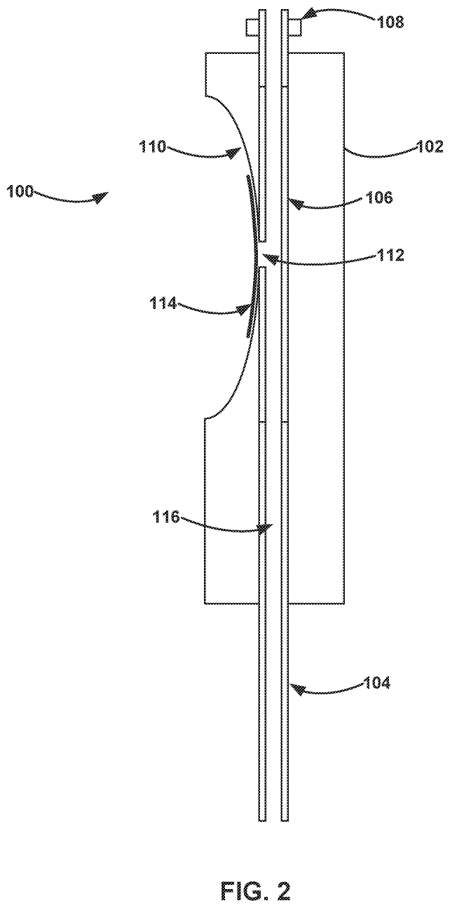

FIG. 2 is a cross-sectional view of an example device 100 configured to position a medical device, such as medical device 30, and check for proper placement within the patient. Device 100 is an example of device 14A of FIG. 1A. Device 100 includes housing 102, shaft 104, luer connection 108, wall 106, and sensing device 114A. Shaft 104 may be similar to shaft 22 of device 14A, housing 102 may be similar to housing 16 of device 14A, aperture 112 may be similar to aperture 20 of device 14A, and luer connection 108 may be similar to luer connection 18 of device 14A.

As shown in the example of FIG. 2, shaft 104, wall 106, and luer connection 108 defines inner passage 116 through housing 102. In this manner, fluid may be drawn through inner passage 116. Aperture 112 is defined by housing 102 and the boundaries of wall 106 in order to enable fluid to pass out from inner passage 116. Wall 106 is shown as being generally linear such that wall 106 is parallel with a straight longitudinal axis of inner passage 116. Sensing device 114A can be placed over aperture 112 to expose a portion of sensing device 114A to the fluid. The outer surface of housing 102 may include curved surface 110 that approaches the portion of the inner passage 116. In this manner, the curved surface of housing 102 may define aperture 112. Curved surface 110 may also enable the outer surface of housing 102 to approach the generally linear path of inner passage 116. In other examples, inner passage 116 may include one or more curves, bends, or varying cross-sectional areas as desired to enable fluid to flow through inner passage 116, through aperture 112, and out of luer connection 108.

In some examples, device 100 may be similar to device 14B of FIG. 1B and include a threaded connector on, or instead of, shaft 104. In any case, device 100 may be configured to couple with other medical devices using coupling mechanisms different than shaft 104 as shown in the example of FIG. 2.

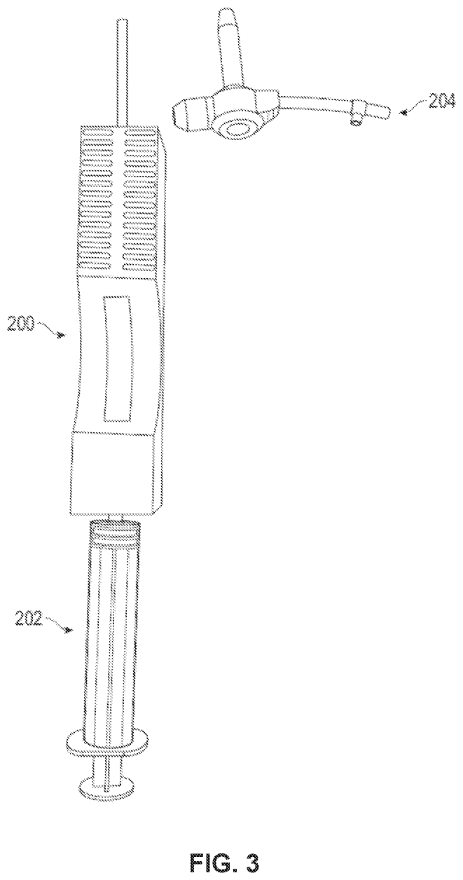

FIG. 3 is a conceptual diagram of an example device 200 in conjunction with a syringe 202 and a low-profile gastrostomy tube 204 for placement within a patient. Device 200 may be similar to device 100 of FIG. 2. Syringe 202 may be similar to syringe 12 of FIG. 1A, and low-profile gastrostomy tube 104 may be similar to medical device 30 of FIG. 1A.

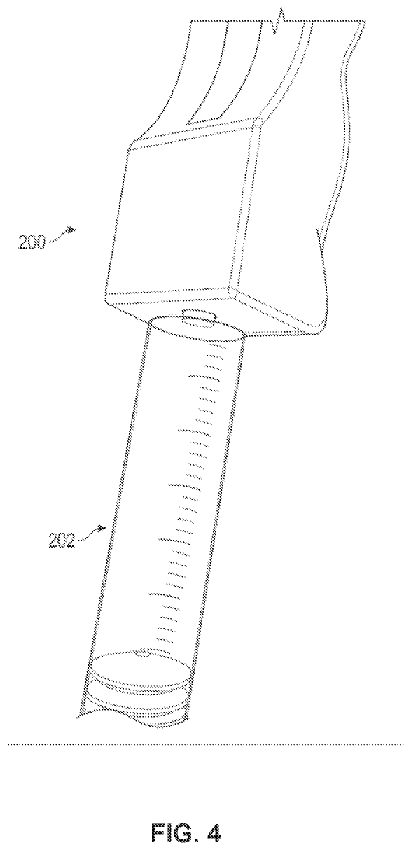

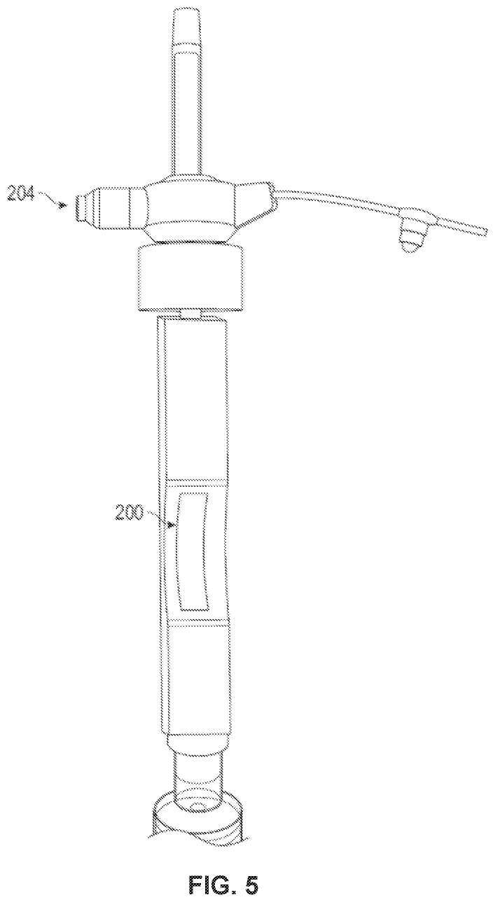

FIG. 4 is a conceptual diagram of a portion of example device 200 and syringe 202 of FIG. 3. FIG. 5 is a conceptual diagram of device 200 of FIG. 3 in the hand of a user, with the shaft of device 200 inserted into a distal portion (e.g., a separate tube or portion of a continuous tube) of low-profile gastrostomy tube 204.

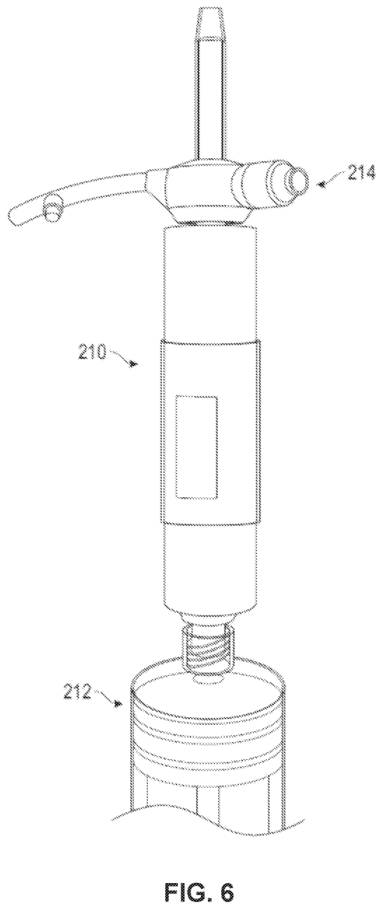

FIG. 6 is a conceptual diagram of an example device 210 attached to syringe 212 and a low-profile gastrostomy tube 214A for placement within a patient. Device 210 may be similar to device 14A of FIG. 1A. Syringe 212 may be similar to syringe 12 of FIG. 1A, and low-profile gastrostomy tube 214A may be similar to medical device 30 of FIG. 1A.

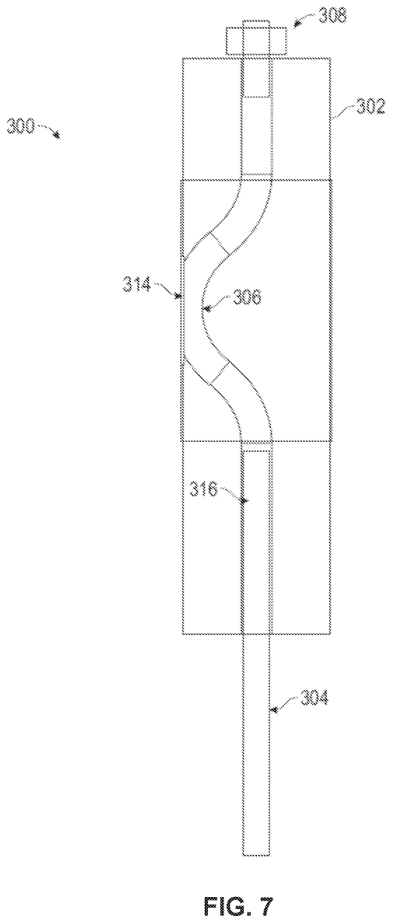

FIG. 7 is a cross-sectional view of example device 300 having a curved inner passage 316. Device 300 is an example of device 14A of FIG. 1A. Device 300 includes housing 202, shaft 304, luer connection 308, wall 306, and sensing device 314A. Shaft 304 may be similar to shaft 22 of device 14A, housing 302 may be similar to housing 16 of device 14A, the aperture (not shown in FIG. 7) may be similar to aperture 20 of device 14A, and luer connection 308 may be similar to luer connection 18 of device 14A.

As shown in the example of FIG. 7, shaft 304, wall 306, and luer connection 308 defines inner passage 316 through housing 302. In this manner, fluid may be drawn through inner passage 316. However, wall 306 is curved and defines the curved inner passage 316. The curvature of inner passage 316 enables device 300 to bring the fluid to the outer surface of housing 302. The aperture is defined by housing 302 and the boundaries of wall 306 in order to enable fluid to pass out from inner passage 316. Sensing device 314A can be placed over the aperture to expose a portion of sensing device 314A to the fluid. Although inner passage 316 is shown with a single curve that curves outward from a central axis of housing 302 to approach the outer surface and the aperture and then curved inward back to the central axis. In other examples, inner passage 316 may include additional curves, bends, or varying cross-sectional areas as desired to enable fluid to flow through inner passage 316, through the aperture, and out of luer connection 308.

In some examples, device 300 may be similar to device 14B of FIG. 1B and include a threaded connector on, or instead of, shaft 304. In any case, device 300 may be configured to couple with other medical devices using coupling mechanisms different than shaft 304 as shown in the example of FIG. 7.

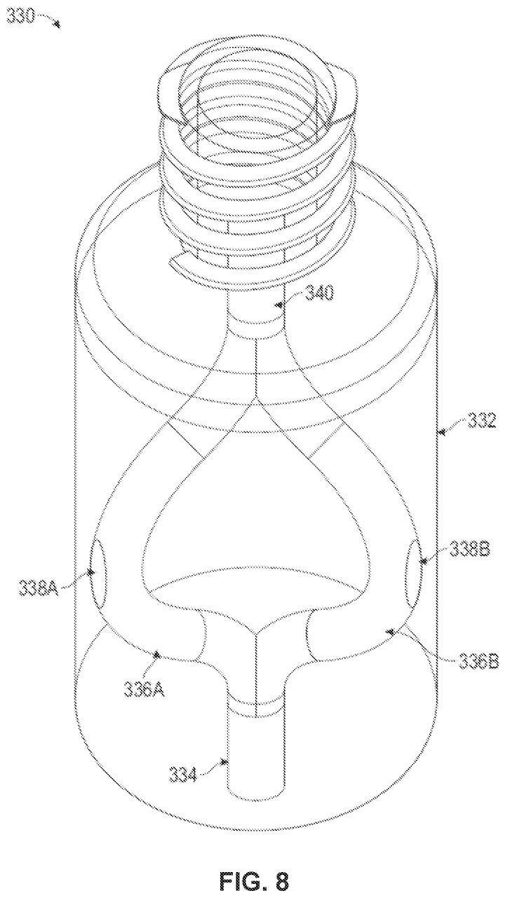

FIG. 8 is a cross-sectional view of an example device 330 having two curved portions of an inner passage in fluid communication with respective apertures at the outer surface of housing 332 of device 330. Device 330 may be similar to device 300 of FIG. 7, or any of devices 14A or 14B, for example. However, device 330 includes an inner passage that separates into two different portions to carry fluid to different apertures at the outer surface of housing 340.

As shown in the example of FIG. 8, housing 332 includes an inner passage (or multiple inner passages) that includes a distal portion 334 and a proximal portion 340. In between distal portion 334 and proximal portion 340, the inner passage splits into separate portions 336A and 336B. Portion 336A of the inner passage curves to the outer surface of housing 332 to define aperture 338A which allows fluid to exit housing portion 336A and housing 332. Similarly, portion 336B of the inner passage curves to the outer surface of housing 332 to define aperture 338B which allows fluid to exit housing portion 336B and housing 332. In this manner, portions 336A and 336B curve in different directions from a central axis to separate the flow of fluid from distal portion 334. Although portions 336A and 336B are shown to have similar curvature, length, and cross-sectional area, portions 336A and 336B may have different curvatures, lengths, cross-sectional areas, or other characteristics that vary between the two.

These two different apertures 338A and 338B enable device 330 to sense multiple samples of the same characteristic of the fluid or different characteristics of the same fluid passing through the inner passage. In this manner, device 330 can provide multiple verification methods for the fluid that is drawn through the inner passage of housing 332. For example, one type of sensing device, such as pH paper, may be positioned and held in place over aperture 338A to detect the characteristic of pH of the fluid. As another example, a different type of sensing device, such as immunochromatographic paper can be placed over aperture 338B to detect pepsin (i.e., an enzyme to break down proteins located within the gastrointestinal tract). In other examples, other enzymes or chemicals may be detected with different types of sensing devices, such as gastric paper or litmus paper. In any case, the detection of multiple characteristics of a target fluid can increase diagnostic accuracy and usefulness of device 330.

In some examples, the outside walls of housing 332, and the walls of portions 336A and 336B (and/or other portions) of the inner passage are constructed of a transparent or semi-transparent material to enable the user to see the fluid flowing within the inner passage. As shown, the inner passage can start as a single lumen and diverge into two (e.g., portions 336A and 336B), where it can impinge on different test substrates (e.g., different sensing devices). After impinging on test substrates, the lumens re-converge into a single outlet (e.g., proximal portion 340) where a syringe can be connected to generate a vacuum and remote fluid as desired.

In other examples, device 330 may include three or more portions to deliver fluid to respective different apertures and sensing devices. In some examples, the different portions of the inner passage may have different cross-sectional areas configured to adjust the fluid flow rate appropriate for the respective sensing device, direct particles of different sizes to different sensing devices, or otherwise modulate the flow of fluid through the different portions of the inner passage in order to detect the different characteristics of the fluid.

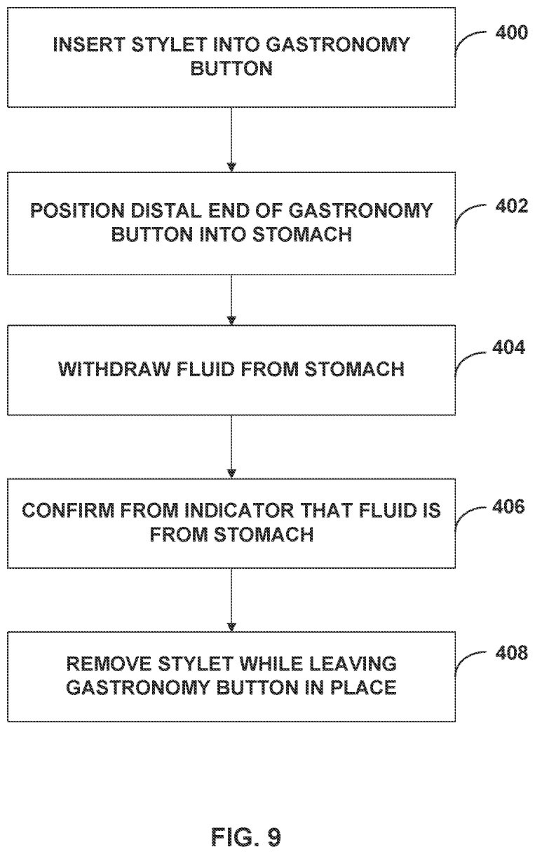

FIG. 9 is a flowchart of an example technique for using device 14A for positioning medical device 30 in a patient, in accordance with one or more techniques of this disclosure. The technique of FIG. 9 will be described with respect to device 14A and system 10A of FIG. 1A, but a similar technique may be used with any other devices or systems describes herein, such as devices 14B, 100, 200, 300, and 330.

As shown in the example of FIG. 9, a user inserts stylet 22 of device 14A into tube 34 of medical device 30, which is a low-profile gastrostomy tube (e.g., a gastrostomy button) in this example (400). The user then manipulates device 14 to move stylet 22 and position the distal end of tube 34 of the gastrostomy button into stomach 42 (402). The user then pulls back on the plunger of syringe 12 to withdraw fluid 44 from stomach 42, through stylet 22, and through the inner passage of device 14A past aperture 20 (404). In this manner, withdrawing the fluid from the stomach can include withdrawing the plunger of syringe 12 coupled to the proximal end of housing 16. The user then confirms, via sensing device 24, that the property of fluid 44 is consistent with contents of stomach 42 (406). In one example, the sensing device 24 may be pH paper, and the user can detect a change in the color of the pH paper after contact with the fluid. In response to determining that fluid 44 is gastric fluid, the user can remove stylet 22 from the gastrostomy button while leaving the gastrostomy button in place (408). In this manner, the user can maintain placement of the gastrostomy button within stomach 42 while removing stylet 22 from tube 34. In some examples, the user can inflate a balloon at the distal end of tube 34 prior to withdrawing stylet 22 from tube 34 in order for the balloon to contact the inner surface of stomach 42.

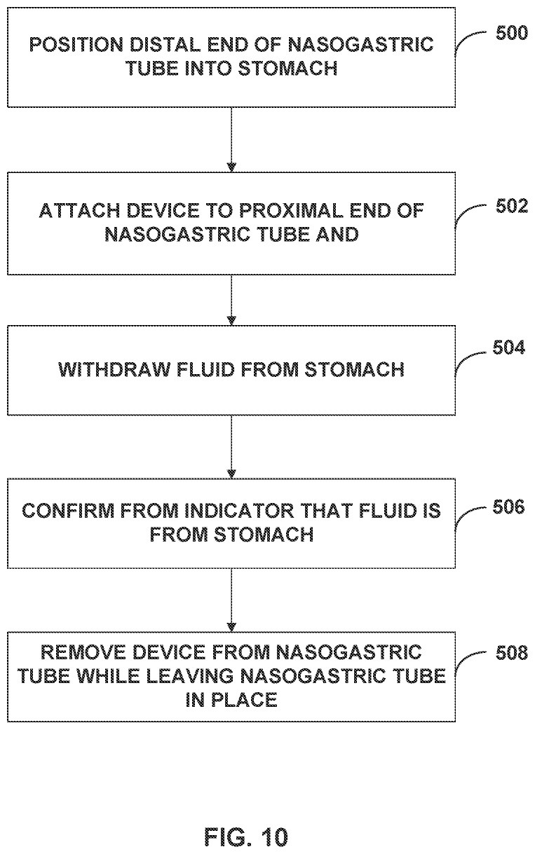

FIG. 10 is a flowchart of an example technique for using device 14B for positioning medical device 50 in a patient, in accordance with one or more techniques of this disclosure. The technique of FIG. 10 will be described with respect to device 14B and system 10B of FIG. 1B, but a similar technique may be used with any other devices or systems describes herein, such as devices 14A, 100, 200, 300, and 330.

As shown in the example of FIG. 10, a user positions tube 54 of medical device 50, such as a nasogastric tube, through the nose and esophagus of the patient such that the distal end of tube 54 is placed into stomach 42 (500). The user than attaches device 14B to the proximal end of medical device 50, such as by screwing threaded connector 52 onto threaded connector 28 (502). The user then pulls back on the plunger of syringe 12 to withdraw fluid 44 from stomach 42, through tube 52 of medical device 50, and through the inner passage of device 14B past aperture 20 (504). In this manner, withdrawing the fluid from the stomach can include withdrawing the plunger of syringe 12 coupled to the proximal end of housing 16. The user then confirms, via sensing device 24, that the property of fluid 44 is consistent with contents of stomach 42 (506). In one example, the sensing device 24 may be pH paper, and the user can detect a change in the color of the pH paper after contact with the fluid. In response to determining that fluid 44 is gastric fluid, the user can remove threaded connector 52 from threaded connector 28 and device 14B from medical device 50 while leaving medical device 50 in place (508). In this manner, the user can maintain placement of the nasogastric tube within stomach 42 while removing device 14B from the proximal end of medical device 50.

The following examples are described herein.

Example 1. A device comprising: a housing defining an outer surface, a first end, and a second end, the housing comprising an inner passage that extends from the first end of the housing to the second end of the housing, wherein the outer surface defines an aperture in fluid communication with a portion of the inner passage; a stylet coupled to the first end of the housing, the stylet defining a channel in fluid communication with the inner passage; and a sensing device configured to cover the aperture, the sensing device configured to detect a property of fluid within the inner passage.

Example 2. The device of example 1, further comprising a cover that retains the sensing device against the aperture.

Example 3. The device of any of examples 1 or 2, wherein the outer surface comprises a curved surface that approaches the portion of the inner passage, the curved surface defining the aperture.

Example 4. The device of any of examples 1 through 3, wherein the inner passage curves outward from a central axis of the housing to approach the outer surface and the aperture.

Example 5. The device of example 4, wherein: the aperture is a first aperture, the sensing device is a first sensing device, and the portion of the inner passage is a first portion, the inner passage comprises the first portion that curves outward in a first direction from the central axis of the housing to approach a first portion of the outer surface and the first aperture, and wherein the inner passage comprises a second portion that curves outward in a second direction from the central axis of the housing to approach a second portion of the outer surface and a second aperture in fluid communication with the second portion of the inner passage, the sensing device is a first sensing device and the property of the fluid is a first property of the fluid, and the device comprises a second sensing device configured to cover the second aperture and detect a second property of the fluid within the second portion of the inner passage.

Example 6. The device of any of examples 1 through 5, wherein the inner passage is defined by one or more inner walls of the housing.

Example 7. The device of any of examples 1 through 6, wherein the housing comprises one or more tubes disposed within an inner wall of the housing, and wherein the one or more tubes define the inner passage.

Example 8. The device of any of examples 1 through 7, wherein the sensing device comprises pH paper.

Example 9. The device of any of examples 1 through 8, wherein the housing comprises a cylindrical shape, and wherein the outer surface is the curved portion of the cylindrical shape.

Example 10. The device of any of examples 1 through 9, wherein the stylet is configured to be inserted into a tube of a gastrostomy button, the stylet being more rigid than the tube of the gastrostomy button.

Example 11. A method comprising: inserting a stylet of a device into an inner passage of a gastrostomy tube, wherein the device comprises: a housing defining an outer surface, a first end, and a second end, the housing comprising an inner passage that extends from the first end of the housing to the second end of the housing, wherein the outer surface defines an aperture in fluid communication with a portion of the inner passage; the stylet coupled to the first end of the housing, the stylet defining a channel in fluid communication with the inner passage; and a sensing device configured to cover the aperture, the sensing device configured to detect a property of fluid within the inner passage; inserting the stylet and a distal end of the gastrostomy tube into a stomach of a patient; withdrawing fluid from the stomach, through the stylet, and through the inner passage of the device past the aperture; and confirming, via the sensing device, that the property of the fluid is consistent with contents of the stomach.

Example 12. The method of example 11, wherein withdrawing the fluid from the stomach comprises withdrawing a plunger of a syringe coupled to the second end of the housing.

Example 13. The method of any of examples 11 or 12, wherein confirming that the property of the fluid comprises detecting a change in a color of pH paper of the sensing device.

Example 14. The method of any of examples 11 through 13, further comprising removing the stylet from the gastrostomy tube while maintaining placement of the gastrostomy tube within the stomach.

Example 15. The method of any of examples 11 through 14, wherein the gastrostomy tube comprises a gastrostomy button.

Example 16. The method of any of examples 11 through 15, wherein the inner passage curves outward from a central axis of the housing to approach the outer surface and the aperture.

Example 17. The method of example 16, wherein: the aperture is a first aperture, the sensing device is a first sensing device, and the portion of the inner passage is a first portion, the inner passage comprises the first portion that curves outward in a first direction from the central axis of the housing to approach a first portion of the outer surface and the first aperture, and wherein the inner passage comprises a second portion that curves outward in a second direction from the central axis of the housing to approach a second portion of the outer surface and a second aperture in fluid communication with the second portion of the inner passage, the sensing device is a first sensing device and the property of the fluid is a first property of the fluid, the device comprises a second sensing device configured to cover the second aperture and detect a second property of the fluid within the second portion of the inner passage, withdrawing fluid from the stomach comprises withdrawing fluid from the stomach, through the stylet, and through the first portion of the inner passage of the device past the first aperture and the second portion of the inner passage of the device past the second aperture, and wherein the method further comprises detecting, via the second sensing device, a second property of the fluid.

Example 18. The method of any of examples 11 through 17, wherein the sensing device comprises pH paper.

Example 19. The method of any of examples 11 through 18, wherein the housing comprises a cylindrical shape, and wherein the outer surface is the curved portion of the cylindrical shape.

Example 20. A system comprising: a device comprising: a housing defining an outer surface, a first end, and a second end, the housing comprising an inner passage that extends from the first end of the housing to the second end of the housing, wherein the outer surface defines an aperture in fluid communication with a portion of the inner passage; a stylet coupled to the first end of the housing, the stylet defining a channel in fluid communication with the inner passage; and a sensing device configured to cover the aperture, the sensing device configured to detect a property of fluid within the inner passage; and a syringe configured to couple to the second end of the housing, wherein the syringe is configured to withdraw fluid through the inner passage and to the aperture in response to withdrawal of a plunger within the syringe.

Example 101. A device comprising: a housing defining an outer surface, a first end, and a second end, the housing comprising an inner passage that extends from the first end of the housing to the second end of the housing, wherein the outer surface defines an aperture in fluid communication with a portion of the inner passage, and wherein the inner passage is curved outward from a central axis of the housing to approach the outer surface and the aperture; a coupling mechanism configured to couple the first end of the housing to a medical device at least partially implantable within a patient; and a sensing device configured to cover the aperture, the sensing device configured to detect a property of fluid within the inner passage.

Example 102. The device of example 1, further comprising a cover that retains the sensing device against the aperture.

Example 103. The device of any one of examples 1 or 2, wherein the outer surface comprises a curved surface that approaches the portion of the inner passage, the curved surface defining the aperture.

Example 104. The device of any one of examples 1 through 3, wherein the coupling mechanism comprises a shaft coupled to the first end of the housing, the shaft defining a channel in fluid communication with the inner passage.

Example 105. The device of any of examples 1 through 4, wherein: the aperture is a first aperture, the sensing device is a first sensing device, and the portion of the inner passage is a first portion, the inner passage comprises the first portion that curves outward in a first direction from the central axis of the housing to approach a first portion of the outer surface and the first aperture, and wherein the inner passage comprises a second portion that curves outward in a second direction from the central axis of the housing to approach a second portion of the outer surface and a second aperture in fluid communication with the second portion of the inner passage, the sensing device is a first sensing device and the property of the fluid is a first property of the fluid, and the device comprises a second sensing device configured to cover the second aperture and detect a second property of the fluid within the second portion of the inner passage.

Example 106. The device of any one of examples 1 through 5, wherein the inner passage is defined by one or more inner walls of the housing.

Example 107. The device of any one of examples 1 through 6, wherein the housing comprises one or more tubes disposed within an inner wall of the housing, and wherein the one or more tubes define the inner passage.

Example 108. The device of any one of examples 1 through 7, wherein the sensing device comprises pH paper.

Example 109. The device of any one of examples 1 through 8, wherein the housing comprises a cylindrical shape, and wherein the outer surface is the curved portion of the cylindrical shape.

Example 110. The device of any one of examples 1 through 9, wherein the medical device is a nasogastric tube, and wherein the coupling mechanism is configured to place the inner passage in fluid communication with a passage of the nasogastric tube.

Example 111. A method comprising: coupling a first end of a device to a nasogastric tube, wherein the nasogastric tube is at least partially implantable within a patient, wherein the device comprises: a housing defining an outer surface, a first end, and a second end, the housing comprising an inner passage that extends from the first end of the housing to the second end of the housing, wherein the outer surface defines an aperture in fluid communication with a portion of the inner passage, and wherein the inner passage is curved outward from a central axis of the housing to approach the outer surface and the aperture; a coupling mechanism configured to couple the first end of the housing to the nasogastric tube; and a sensing device configured to cover the aperture, the sensing device configured to detect a property of fluid within the inner passage; withdrawing fluid from the stomach, through the nasogastric tube, and through the inner passage of the device past the aperture; and confirming, via the sensing device, that the property of the fluid is consistent with contents of the stomach.

Example 112. The method of example 11, wherein withdrawing the fluid from the stomach comprises withdrawing a plunger of a syringe coupled to the second end of the housing.

Example 113. The method of any one of examples 11 or 12, wherein confirming that the property of the fluid comprises detecting a change in a color of pH paper of the sensing device.

Example 114. The method of any one of examples 11 through 13, further comprising removing the device from the nasogastric tube while maintaining placement of the nasogastric tube within the stomach.

Example 115. A system comprising: a device comprising: a housing defining an outer surface, a first end, and a second end, the housing comprising an inner passage that extends from the first end of the housing to the second end of the housing, wherein the outer surface defines an aperture in fluid communication with a portion of the inner passage, and wherein the inner passage is curved outward from a central axis of the housing to approach the outer surface and the aperture; a coupling mechanism configured to couple the first end of the housing to a medical device at least partially implantable within a patient; and a sensing device configured to cover the aperture, the sensing device configured to detect a property of fluid within the inner passage; and a syringe configured to couple to the second end of the housing, wherein the syringe is configured to withdraw fluid through the inner passage and to the aperture in response to withdrawal of a plunger within the syringe.

Various examples have been described. These and other examples are within the scope of the following claims.

What is claimed is:

1. A device comprising:
a housing defining an outer surface, a first end, and a second end, the housing comprising an inner passage that extends from the first end of the housing to the second end of the housing, wherein the outer surface defines an aperture in fluid communication with a portion of the inner passage;
a stylet coupled to the first end of the housing, the stylet defining a channel in fluid communication with the inner passage; and
a sensing device configured to cover the aperture, the sensing device configured to detect a property of fluid within the inner passage.

2. The device of claim 1, further comprising a cover that retains the sensing device against the aperture.

3. The device of claim 1, wherein the outer surface comprises a curved surface that approaches the portion of the inner passage, the curved surface defining the aperture.

4. The device of claim 1, wherein the inner passage curves outward from a central axis of the housing to approach the outer surface and the aperture.

5. The device of claim 4, wherein:
the aperture is a first aperture, the sensing device is a first sensing device, and the portion of the inner passage is a first portion,
the inner passage comprises the first portion that curves outward in a first direction from the central axis of the housing to approach a first portion of the outer surface and the first aperture, and wherein the inner passage comprises a second portion that curves outward in a second direction from the central axis of the housing to approach a second portion of the outer surface and a second aperture in fluid communication with the second portion of the inner passage,
the sensing device is a first sensing device and the property of the fluid is a first property of the fluid, and
the device comprises a second sensing device configured to cover the second aperture and detect a second property of the fluid within the second portion of the inner passage.

6. The device of claim 1, wherein the inner passage is defined by one or more inner walls of the housing.

7. The device of claim 1, wherein the housing comprises one or more tubes disposed within an inner wall of the housing, and wherein the one or more tubes define the inner passage.

8. The device of claim 1, wherein the sensing device comprises pH paper.

9. The device of claim 1, wherein the housing comprises a cylindrical shape, and wherein the outer surface is the curved portion of the cylindrical shape.

10. The device of claim 1, wherein the stylet is configured to be inserted into a tube of a gastrostomy button, the stylet being more rigid than the tube of the gastrostomy button.

11. A method comprising:
inserting a stylet of a device into an inner passage of a gastrostomy tube, wherein the device comprises:
a housing defining an outer surface, a first end, and a second end, the housing comprising an inner passage that extends from the first end of the housing to the second end of the housing, wherein the outer surface defines an aperture in fluid communication with a portion of the inner passage;
the stylet coupled to the first end of the housing, the stylet defining a channel in fluid communication with the inner passage; and
a sensing device configured to cover the aperture, the sensing device configured to detect a property of fluid within the inner passage;
inserting the stylet and a distal end of the gastrostomy tube into a stomach of a patient;
withdrawing fluid from the stomach, through the stylet, and through the inner passage of the device past the aperture; and
confirming, via the sensing device, that the property of the fluid is consistent with contents of the stomach.

12. The method of claim 11, wherein withdrawing the fluid from the stomach comprises withdrawing a plunger of a syringe coupled to the second end of the housing.

13. The method of claim 11, wherein confirming that the property of the fluid comprises detecting a change in a color of pH paper of the sensing device.

14. The method of claim 11, further comprising removing the stylet from the gastrostomy tube while maintaining placement of the gastrostomy tube within the stomach.

15. The method of claim 11, wherein the gastrostomy tube comprises a gastrostomy button.

16. The method of claim 11, wherein the inner passage curves outward from a central axis of the housing to approach the outer surface and the aperture.

17. The method of claim 16, wherein:
the aperture is a first aperture, the sensing device is a first sensing device, and the portion of the inner passage is a first portion,
the inner passage comprises the first portion that curves outward in a first direction from the central axis of the housing to approach a first portion of the outer surface and the first aperture, and wherein the inner passage comprises a second portion that curves outward in a second direction from the central axis of the housing to approach a second portion of the outer surface and a second aperture in fluid communication with the second portion of the inner passage,
the sensing device is a first sensing device and the property of the fluid is a first property of the fluid,
the device comprises a second sensing device configured to cover the second aperture and detect a second property of the fluid within the second portion of the inner passage,
withdrawing fluid from the stomach comprises withdrawing fluid from the stomach, through the stylet, and through the first portion of the inner passage of the device past the first aperture and the second portion of the inner passage of the device past the second aperture, and
wherein the method further comprises detecting, via the second sensing device, a second property of the fluid.

18. The method of claim 11, wherein the sensing device comprises pH paper.

19. The method of claim 11, wherein the housing comprises a cylindrical shape, and wherein the outer surface is the curved portion of the cylindrical shape.

20. A system comprising:
a device comprising:
a housing defining an outer surface, a first end, and a second end, the housing comprising an inner passage that extends from the first end of the housing to the second end of the housing, wherein the outer surface defines an aperture in fluid communication with a portion of the inner passage;
a stylet coupled to the first end of the housing, the stylet defining a channel in fluid communication with the inner passage; and
a sensing device configured to cover the aperture, the sensing device configured to detect a property of fluid within the inner passage; and
a syringe configured to couple to the second end of the housing, wherein the syringe is configured to withdraw fluid through the inner passage and to the aperture in response to withdrawal of a plunger within the syringe.
